(19) United States
(12) Patent Application Publication
(10) Pub. No.: US 2006/0034849 A1
(43) Pub. Date: Feb. 16, 2006

(54) Antibodies reactive with beta (1-3)-glucans

(76) Inventors: Inger Mattsby-Baltzer, Goteborg (SE); Nahid Kondori, Molndal (SE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/532,055

(22) PCT Filed: Oct. 21, 2003
(86) PCT No.: PCT/SE03/01638

§ 371 Date: Apr. 21, 2005

(30) Foreign Application Priority Data
Oct. 21, 2002 (SE) 0203116-9

(51) Int. Cl. A61K 39/395; C07K 16/14

(52) U.S. Cl. 424/152.1; 530/388.5

(57) ABSTRACT

Monoclonal antibodies reactive with β(1-3)-glucans are disclosed. More precisely, two monoclonal antibodies, B3B and A10A, reactive with β(1-3)-glucan and/or β(1-3) (1-6)-glucan associated epitopes in free, non-associated form, and/or in cell wall fragments of Candida and Cryptococcus are disclosed. Further, A10A is also reactive with a β(1-6) (1-3)-glucan epitope present on the intact cell surface of C. albicans, C. parapsilosis, C. krusei, C. glabrata and/or C. neoformans. The antibodies can be used for the detection of free, cell wall associated, and/or cell surface associated β(1-3) glucans utilizing immunoassays or immunohistology for the laboratory diagnosis of fungal infections. They may be used also for detection of airborn mould, or mould present in dust, water, or in any other component.

### TECHNICAL FIELD

The present invention relates to new antibodies reactive with β(1-3)-glucans, as well as to the use of such antibodies.

### BACKGROUND OF THE INVENTION

Fungal infections may occur at many places in the human or animal body, e.g. in the vagina or in the oral cavity.

Invasive fungal infections are increasing because of the growing number of immunocompromised patients (7). Almost all of these infections occur in critically ill patients suffering from an underlying disease.

In Candida species which are the most common fungi isolated from patients with invasive fungal infection, the yeast cells are surrounded by a rough, rigid cell wall that represents 20-25% of the dry weight of the cells (9). The cell wall of C. albicans and S. cerevisiae consists of about 85-90% polysaccharide, 10-15% protein, and a small amount of lipids (19, 20). The polysaccharide components consist of mannan, glucan, and a small amount of chitin. Most of the proteins are covalently linked to the mannan (mannoprotein), which is located in the outermost layer of the cell wall. A fraction of the proteins is also covalently linked to glucan (8). The proportions of these different components vary with the species, but in S. cerevisiae there are approximately equal proportions of mannan and glucan, and about equal amounts of alkali-soluble glucan and alkali-insoluble glucan (3). The glucan microfibriles are located mostly in the inner part of the cell wall. The high mannose content present in C. albicans cell wall is absent in C. neoformans, and glucose is the major monosaccharide constituent of the cryptococcal cell wall. The cell wall of uncapsulated C. neoformans is composed mainly of glucan. β(1-3)-glucans are unique for all medically important fungi and are shed during growth (16). Thus, determination of β(1-3)-glucans appear to be a useful marker in the laboratory diagnosis of deep fungal infections.

The analysis of β(1-3)-glucans is based on the binding of the polysaccharide to the coagulation factor G. This glucan test, however, has some limitations. It does not react exclusively with β(1-3)glucans, since also (1-3) (1-4)-α-D-glucan (negaran) and (1-2) (1-3) (1-6)-α-D-glucan (yeast α-D-mannan), and (1-6)-β-D-glucan (gyrophoran) may activate the G factor (18). The reactivity of factor G is also dependent on the molecular weight, conformation and degree of branching of the glucans (18). Moreover, there are some contradictions regarding its effectiveness of determining glucans in Cryptococcus neoformans infections (17).

Compounds with a high binding specificity for β(1-3)-glucans would be useful tools for providing an analysis of β(1-3)-glucans in any body fluid, such as blood, urine, or in biopsy specimens of patients with suspected fungal infections, and consequently for providing a laboratory diagnosis of fungal infection.

### SUMMARY OF THE INVENTION

The object of the present invention is to provide antibodies reactive with β(1-3)-glucans as present in free form, in cell wall fragments, and in intact fungal cells with a high specificity for use in the laboratory diagnosis of fungal infections.

According to a first aspect of the present invention, a monoclonal antibody reactive with β(1-3)-glucans is provided. Said antibody is reactive with β(1-3) and/or β(1-3) (1-6)-glucan associated epitopes in free, non-associated form, in cell wall fragments and/or on an intact cell surface. Said antibody is B3B or A10A.

According to a second aspect of the invention, the use of said antibody for the diagnosis of fungal infections is provided. Further, said antibody may be used for the detection of medically important fungi, e.g. mould, in air, water, dust or other components.

According to a third aspect of the invention, a diagnostic kit for the diagnosis of fungal infections comprising said monoclonal antibody is provided.

According to a fourth aspect of the present invention, a method for diagnosing fungal infections comprising said monoclonal antibody is provided. Additionally, a method for detecting medically important fungi, e.g. mould, is provided.

### BRIEF DESCRIPTION OF THE DRAWINGS

FIG. 1 shows the antibody activities of A10A and B3B to β(1-3)(1-6)-glucan, β(1-3)-glucan, β(1-3)(1-4)glucan, β(1-6)-glucan, Candida albicans cell wall fragments (CaCW), and Cryptococcus neoformans cell wall fragment (CnCW) as analyzed by ELISA at a dilution of 1/10. The antibody activity is expressed as the absorbance value.

### DETAILED DESCRIPTION OF THE INVENTION

As stated above, the cell wall of all medically important fungi contains a unique polyglucose compound, a β(1-3)-glucan. β(1-3)-glucans refer to polysackarides having the basic unit β(1-3). These glucans may be β(1-3)-glucans without side chains, or may be branched to various degrees having β(1-6) side chains, β(1-3)(1-6)glucans. The side chains may be varied with respect to the number of β(1-6) per β(1-3), the length of β(1-6)branched β(1-3) etc.

Murine monoclonal antibodies were produced against linear β(1-3)-glucans and β(1-6)-branched β(1-3)-glucans (also called β(1-3)(1-6)-glucan) and their specificity was characterized. The antibodies were analysed for reactivity to other β-glucans, fungal cell wall fragments, and intact fungal cells.

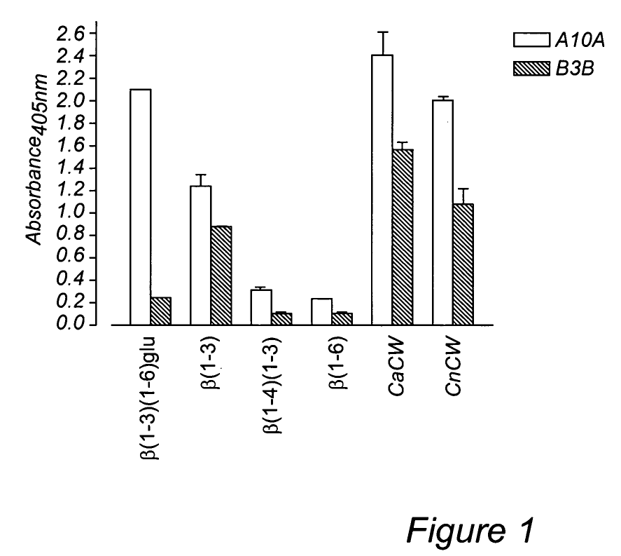

Two monoclonal antibodies, A10A and B3B, reactive with β(1-3)-glucan and β(1-3)(1-6)-glucan in ELISA, recognized immunoreactive epitopes in Candida albicans and non-encapsulated Cryptococcus neoformans cell wall fragments (CaCW, CnCW) (FIG. 1). The A10A epitope was present in both β(1-3)-glucan and β(1-6)-glucan. The B3B epitope included β(1-3)-glucan, but most probably not β(1-6)glucan. Thus, B3B appeared to recognize the β(1-3)-linkage, present in β(1-3)-glucan and β(1-3)(1-6)-glucan, while A10A reacts with glucan consisting of both types of linkages, i.e. reacts with β(1-3)-glucans, β(1-6)-glucans and β(1-3)(1-6)-glucans.

By indirect immunofluorescence only A10A recognized a β(1-3)(1-6) associated epitope on the intact cell surface of C. albicans, C. parapsilosis, C. krusei, C. glabrata, and C. neoformans.

In summary, B3B demonstrated the presence of immunoreactive epitopes, i.e. β(1-3)-glucan and β(1-3)(1-6)glucan, in cell wall fragments of C. albicans and C. neoformans (FIG. 1), as well as in free form β(1-3)- and β(1-3)(1-6)-glucans (table 2), while A10A in addition recognized a β(1-3)(1-6)-glucan associated epitope that was readily available on the surface of whole cells of C. neoformans and all Candida species tested.

Thus, the two monoclonal antibodies to β(1-3)glucans, A10A and B3B, could be used in combination (although not excluding also separately) in an immunoassay for the detection of free, cellwall associated or cell surface associated β(1-3)-glucans. Thereby, they are of help in laboratory diagnosis of fungal infections, in particular deep fungal infections, but also superficial infections, such as Candida vaginitis or mucocutane candidiasis.

Thus, in the research work leading to the present invention murine monoclonal antibodies directed against β(1-6)-branched β(1-3)-glucans were characterized by ELISA with respect to crossreactions with β(1-3)-, β(1-6)-, β(1-4)(1-3)-glucans, C. albicans and C. neoformans cell wall fragments. The presence of a β glucan epitope on the surface of the cell wall of C. albicans, C. parapsilosis, C. glabrata, C. krusei, an uncapsulated mutant of C. neoformans was investigated by immunofluorescence microscopy.

We present what to our knowledge is the first mAb (A10A) that reacts with a β(1-3)(1-6)-glucan epitope on the intact cell surface of Candida.

By a β(1-3)-glucan associated epitope is meant an epitope which is present in β(1-3)-glucans, and β(1-3)(1-6)-glucans.

By a (1-3)(1-6)-glucan associated epitope is meant an epitope which is present in β(1-3)-glucans, β(1-6)glucans, and β(1-3)(1-6)-glucans.

Materials and Methods

Strains and Condition of Growth

C. albicans ATCC 64549, C. glabrata ATCC 90030, C. parapsilosis CCUG 37233, C. krusei ATCC 6258, and an uncapsulated C. neoformans strain 602 were cultivated in Sabouraud dextrose broth, at 37° C. overnight. The conversion of yeast to germ tube and hyphal forms of C. albicans was carried out by transferring the C. albicans yeast cells to RPMI 1640 and cultivation at 37° C. for 18 h.

Antigens

Cell Wall Fragments

Cell wall fragments of C. albicans (CaCW) and C. neoformans strain 602 (CnCW) were prepared by treatment of the yeast cells by glass beads as described earlier (12). The glucan structure in CaCW is composed of branching β(1-3)(1-6) linkages. The cell wall of uncapsulated C. neoformans is composed mainly of α(1-3)(1-4)D and β(1-3)(1-6)-glucans (6).

Glucans

Glucan from Saccharomyces cerevisiae with β(1-6)-branched β(1-3)-linked glucose residues [β(1-3)(1-6)glu], Alcaligenes faecalis curdlan with (1-3)-β-linkages [β(1-3)], and glucan from barley with (1-4)(1-3)-β-linkages [β(1-4)(1-3)] were purchased from Sigma (St Louis, USA). Pustulan from lichen Umbilicaria papullosa with (1-6)-β-linked glucose residues [β(1-6)] was purchased from Calbiochem (San Diego, USA). According to the manufacturer pustulan contained only glucose. The purity of the glucans of baker yeast, curdlan, and barley were 98, 99 and 96% respectively, according to the specifications. Table 1 summarizes the trivial names, physical properties, and sources of the β-glucans used in this study. β(1-3)(1-6)glu, β(1-4)(1-3), and β(1-3) were dissolved in 0.3M NaOH at a concentration of 20 mg/ml. β(1-6) was dissolved in water at 100° C. at a concentration of 20 mg/ml.

TABLE 1
Structural and physical properties of β-glucans used in this study

| Trivial | Type of | | Molecular | Solubility | Linear/ |
|---|---|---|---|---|---|
| name | linkages | Source | weight | in water | branched |
| Yeast | β(1-3)(1-6)-D- | Saccharomyces | 17,000 | insoluble | branched |
| glucan | | cerevisiae | | | |
| Curdlan | β(1-3)-D- | Alcaligenes | 294,000 | insoluble | linear |
| | | faecalis | | | |
| Barley | β(1-4)(1-3)-D- | Barley plant | 23,000 | insoluble | linear |
| Pustulan | β(1-6)-D- | Umbilicaria | 20,000 | soluble | linear |
| | | papullosa | | | |

Antibodies to β-Glucan
Production of mAbs

For the production of mAbs female Balb/c mice (6-8 weeks old) were immunized intraperitoneally (i.p) with either 50 μg of β(1-3) (2 mice), β(1-3) (1-6)glu (4 mice) or 2.5×10⁷ cells of formaldehyde treated uncapsulated C. neoformans (4 mice) suspended in 200 μl PBS containing 1 μg of cholera toxin, which was used as an adjuvant (23). Two and four weeks later, the mice received intraperitoneal injections with the same amount of antigen. One week after the last injection, blood was collected and the antibody response to β(1-3)(1-6)glu analyzed by ELISA. After an additional week another injection with the same amount of antigen was given, and three to four days later the animals were killed and their spleens used for fusion.

Myeloma cells were cultured in Iscoves medium supplemented with 2 mM L-glutamine, penicillin (100 U/ml), streptomycin (100 μg/μl) and 1% (w/v) fetal bovine serum (growing medium). Cell fusion and selection of hybrids were carried out as described by Köhler and milstein (11). Spleen lymphocytes from immunized mice were fused with SP2/0 murine myeloma cells at a 5:1 ratio using PEG 1500 (Boehringer Mannheim GmbH, Mannheim, Germany) as the fusing agent. The fused cells were distributed in 96-well culture plates at an approximately density of 4×10⁵ cells in 200 μl HAT selection medium (growing medium supplemented with hypoxanthin, aminopterine, thymidine). On day 10 post-fusion, the culture supernatants were screened for the presence of antibodies specific to β(1-3)(1-6)glu and β(1-3) by ELISA. Positive hybridomas, which all were of IgM isotype as determined by ELISA, were cloned by limiting dilution on a feeder layer of Balb/c peritoneal macrophages. Cells were grown in HAT medium for two weeks. The HAT was substituted by HT medium (growing medium supplemented with hypoxanthin and thymidine). Positive clones were cultivated in serum free medium HyQ-CCM1 (from Hyclone Laboratories Inc, Utah, USA).

MAbs were purified by (NH₄)₂SO₄ precipitation followed by affinity chromatography on agarose gel with covalently linked IgG goat anti-mouse IgM (Sigma, St Louis, USA). The fraction was dialyzed against PBS overnight at 4° C. The protein concentration was determined by Coomassie protein assay reagent kit (Pierce, Ill.; USA).

The protein concentration was adjusted to 100 μg/ml in 1% BSA in PBS and stored −70° C.

ELISA

Microplate wells (Nunc immunoplate, Denmark) were coated with 100 μl of a 50 μg/ml solution of β(1-3), β(1-4)(1-3), β(1-6), CaCW or CnCW and a 20 μg/ml of β(1-3)(1-6)glu solution containing 50 mM Na₂CO₃ buffer, pH 9.3. The plates were incubated at room temperature (r.t.) for two hours and thereafter kept at 4° C. overnight. After rinsing the plate once with PBS, 100 μl of blocking buffer (BF) (1% BSA in PBS) were added to each well and the plate incubated for 1 h at r.t. The plate was rinsed once with PBS. mAbs diluted in 1/10, 1/50, 1/100 and 1/1000 in PBS, were added to each well (100 μl) and incubated for two hr at r.t. Hereafter the plate was rinsed three times with 0.05% Tween-20 in PBS (PBS-T) between each incubation step. Biotinylated rabbit anti-mouse IgM (DAKO, Glostrup, Denmark) diluted 1/5000 in PBS-T was added to the wells (100 μl). The plate with monoclonal antibodies was further incubated at r.t. for 2 h, and thereafter 100 μl of alkaline phosphatase conjugated extravidin (Sigma, St Louis, USA) diluted 1/1000 in PBS-T were added and the plate was incubated at r.t. for 60 min. Paranitrophenylphosphate (1 mg/ml, Sigma, St Louis, USA) diluted in diethanolamine buffer (pH 9.8) was added to each well and the absorbance was read at 405 nm when a suitable color had developed.

Inhibition-ELISA

Increasing amounts of β(1-3)(1-6)glu, β(1-3), β(1-4)(1-3), β(1-6), CaCW, CnCW (1-1000 μg/ml) were added to series of tubes containing a constant amount of mAb or rabbit serum. The mAbs were also incubated with monosaccharide; β-D-glucose, glucose amine and mannose or disaccharides; trehalose with α(1-1), maltose with α(1-4) and cellobiose with β(1-4) linkages at the concentration of 50 and 1000 μg/ml. The mAb solutions were incubated at r.t. for 30 min and kept at 40C overnight. The solutions were centrifuged to remove any precipitates, and the supernatants were analyzed for the remaining antibody activity against CaCW or β(1-3)(1-6)glu, as antigens. The mAb A10A and B3B were diluted 1/50 and 1/20 respectively, in PBS supplemented with 0.1% BSA for the inhibition assay. The inhibition capacity of an antigen was defined as the concentration needed for inhibiting the antibody activity to 50%, i.e. reducing the absorbance to 50% of that of the unabsorbed serum dilution (EI₅₀) (14).

Immunofluorescence Microscopy (IF)

The immunofluorescence assay was carried out as described by Casanova et.al. with some modifications (2). Microorganisms were washed 3 times in PBS, the concentration of the cells were adjusted to 10⁶ cells/ml in PBS and drops of the cell suspensions were placed on microscope slides and allowed to air dry. The microorganisms were fixed for 20 min with 0.2% formaldehyde in PBS. The microscope slides were washed in 3 changes of PBS for a total of 15 min. MAbs (20 μl) diluted 1/20 in PBS, were added to the slides and were incubated at r.t. for 60 min in a moister chamber. The slides were washed as described above. Biotin conjugated rabbit anti-mouse IgM (DAKO, Glostrup, Denmark) diluted 1/100 in PBS was added and slides were incubated at r.t for another 60 min. FITC-conjugated avidin (Sigma, St Louis, USA) diluted 1/200 in PBS was added (20 μl) to the slides and were incubated at r.t. for 30 min in a moister chamber. The slides were washed as above and rinsed with distilled water, and mounted with Kaiser's glycerol gelatin (Merck, Darmstadt, Germany). The cells were examined with a Zeiss photomicroscope equipped with fluorescence.

### EXAMPLES

### Example 1

### Specificity of mabs against β(1-3)-glucans

MAbs were screened against β(1-3)(1-6)glu and β(1-3). Only mAbs of IgM class were found. Out of four selected mAbs two were further analyzed. The reactivity of A10A and B3B against β(1-3)(1-6)glu and β(1-3), β(1-4)(1-3), β(1-6), CnCW, and CaCW were studied (FIG. 1). A10A showed a high antibody activity against all antigens except for β(1-6), and β(1-4)(1-3). B3B showed an overall lower activity against the antigens. The highest antibody activity was obtained against CaCW followed by CnCW. It was intermediate against β(1-3) and low against β(1-3)(1-6)glu, while it was not active against β(1-6) and β(1-4)(1-3). The highest antibody activity for both mAbs was found against CaCW. In addition, A10A showed a high activity against β(1-3)(1-6)glu.

The cross-reaction between β(1-3)(1-6)glu or CaCW and the various glucan antigens were studied by inhibition-ELISA.

It was found that the EI₅₀ of A10A for the homologous antigen, β(1-3)(1-6)glu, and CnCW were almost identical (6 and 5 μg/ml, respectively) (Table 1). EI₅₀ for β(1-3) and β(1-6) was 7-fold higher. EI₅₀ for β(1-4) (1-3) and CaCW was more than 60 and 40 times higher respectively, than the β(1-3)(1-6)glu or CnCW.

TABLE 1
Inhibition of the anti-β(1-3)(1-6)glu and CaCW antibody activities
of A10A by absorption with β(1-3)(1-6)glu, β(1-3), β(1-4)(1-3),
β(1-6), CaCW, and CnCW. A10A was diluted 1/50

EI₅₀ (μg/ml) ± Standard deviation

| Absorbing agent | β(1-3)(1-6)glu^{#} | CaCW |
|---|---|---|
| β(1-3)(1-6)glu | 6 ± 2 | 31 ± 12 |
| β(1-3) | 40 ± 12 | 185 ± 170 |
| β(1-4)(1-3) | 359 ± 39 | >* |
| β(1-6) | 43 ± 17 | >* |
| CaCW | 238 ± 112 | 56 ± 6 |
| CnCW | 5 ± 2 | 6 ± 4 |

^{#}The absorbance value of the unabsorbed antibody was 1.8 against β(1-3)(1-6)glu and 1.2 against CaCW.
*No inhibition at the highest concentration tested, 1000 ug/ml.

The A10A activity against CaCW showed that CnCW was a 9-fold stronger inhibitor than the homologous antigen (Table 1). In addition, β(1-3)(1-6)glu was also stronger as inhibitor than CaCW. EI₅₀ for CaCW was almost twofold higher (56 μg/ml) than that of β(1-3)(1-6)glu (31 μg/ml). Thus, the A10A epitope involved the branching region of the glucan, the β(1-3)(1-6) linkage, which was available to a higher extent in CnCW than in CaCW.

The specificity of B3B to CaCW was analyzed by inhibition-ELISA. The EI₅₀ for CaCW and β(1-3) was roughly the same and they were more than 15 times higher that of EI₅₀ for β(1-3)(1-6)glu (Table 2). β(1-4)(1-3) and CnCW did not inhibit the anti-CaCW antibody activity at the highest concentration tested. The EI₅₀ for β(1-6) was almost 40-fold higher than that of β(1-3)(1-6)glu. This inhibition pattern of B3B differed from that of A10A by the lack of inhibitory effect of CnCW, while still being inhibited by β(1-3)(1-6)glu. Thus, the B3B epitope was highly exposed by the free form of β(1-3)(1-6). None of the mono- and disaccharides inhibited the anti-β(1-3)(1-6)glu antibody activity of the two mAbs.

TABLE 2
Inhibition of the B3B anti- CaCW antibody activity by absorption
with β(1-3)(1-6)glu, β(1-3), β(1-4)(1-3),
β(1-6), CaCW, and CnCW. The mAb was diluted 1/20
in in PBS containing 0.1% BSA. The absorbance value was
0.4 of the unabsorbed antibody.

| Absorbing agent | EI₅₀ (μg/ml) |
|---|---|
| β(1-3)(1-6)glu | 20 |
| β(1-3) | 450 |
| β(1-4)(1-3) | >* |
| β(1-6) | 750 |
| CaCW | 306 |
| CnCW | >* |

*>, no inhibition at the highest concentration tested (1000 μg/ml).

### Example 2

### Availability of β(1-3) (1-6) epitopes on the cell surface of Candida and C. neoformans

The availability of β(1-3)(1-6)-glucan for antibody binding on the cell surface of various Candida species and C. neoformans were analyzed by IF microscopy using A10A and B3B. Yeast and mycelial forms of C. albicans, C. parapsilosis, C. krusei, C. glabrata and the uncapsulated mutant of C. neoformans were all positive by IF (FIG. 3). The intensity of fluorescence differed depending on the morphology and distribution of the antigens in the cell wall. Uncapsulated C. neoformans was strongly immunoreactive with A10A. This mAb also stained the yeast and mycelial forms of C. albicans, but somewhat weaker. The other species of Candida, C. parapsilosis, C. krusei and C. glabrata were all stained with A10A.

B3B did not stain any of the fungal strains.

Discussion

While A10A reacted with an epitope exposed on the cell surface of intact fungal cells, the other one recognized an epitope present in the cell wall fragments only. Both mabs reacted with the free form β(1-3) or β(1-3) (1-6)-glucan. The mycelial form of C. albicans was stained with A10A to the same degree as the yeast form as shown by IF.

The novel A10A mAb also recognized an epitope present in β(1-6). MAbs directed against β(1-6) and β(1-3)(1-6)-glucans have been described earlier (4, 22). The mAb directed against β(1-6) as obtained by immunization with zymolyase extract from C. albicans was shown not to react with epitopes on the cell wall of C. albicans unless the outer layer, being formed by mannoproteins, was disrupted by the effect of tunicamycin. Tunicamycin interferes with the N-glycosylation of proteins so that new synthesis of mannoproteins will not become glycosylated during cell growth (10). Thus, A10A as well as B3B differed from this β(1-6) epitope binding monoclonal. Regarding the other two reported mAbs, one was suggested to be mainly directed against β(1-3) in Schizophyllan (β(1-3)-glucan with branching β(1-6) glucose residues) (5), while the detailed specificity of the other one with regard to various glucans was not reported. The presence of those epitopes on the cell surface of fungi was, however, not studied. The mAbs were produced for measuring either schizophyllan in serum during treatment by this agent as an anti-cancer drug, or for determining the immunological properties of another anti-tumor polysaccharide containing β(1-3) and β(1-6)-glucans (5). Thus, in two of the first described monoclonals none was analysed for binding activities against native β(1-3)glucans exposed in the cell wall fragments of Candida or Cryptococcus as was shown for A10A and B3B. In order to perform analyses on natural components such as intact cells, cell wall fragments, or shed β(1-3) glucans, only our mabs are characterized with respect to this.

Our second mAb B3B did not recognize cell wall antigens in indirect IF and only weakly in inhibition-ELISA. Most probably the explanation for this is the presence of the epitope mainly in the deeper parts of the cell wall and thereby not available on the cell surface of the intact cell. Yet another explanation could be that it only recognizes a particular form of the glucan antigen, since the weak anti-CaCW activity of B3B was inhibited by β(1-3) (1-6)-glucan at a low concentration. Although B3B was produced against β(1-3), the EI₅₀ for this glucan regarding B3B anti-CaCW activity was approximately 15 times higher that of β(1-3) (1-6). The fact that β(1-3) is linear and β(1-3)(1-6) is branched in addition to a 10 times higher molecular weight than β(1-3) (1-6) may have influenced the epitope density. It is also known that the ultrastructure of higher molecular weight β glucans exhibits various forms such as single-helical, triple-helical, and a mixture of both, due to interchain hydrogen bonding between each main chain of polyglucose residues (25). Lower molecular weight P glucans adopt a randomly coiled form in aqueous solution (1). The percentage of branching, i.e. the number of (1-6)-per (1-3)-linkage may also differ between different fungal species. The availability of epitopes may be higher in randomly coiled regions of branched β glucans.

During growth medically important fungi seem to shed β(1-3)-glucan into the culture medium. The concentration of β(1-3)-glucan in serum from patients with deep fungal infections can be very high as determined by the G factor based Limulus assay (16, 17, 21). We have found β(1-3)glucan in serum of all patients with candidemia, but in none of women with superficial Candida infection, or healthy controls (13). Thus, β(1-3)-glucan seems to be a sensitive assay. However, since also other types of glucans may activate the Limulus assay (24b) an immunoassay based on two specific antibodies would be more specific. Two assays have been reported for the determination of β(1-3) glucan levels: the first utilizes a monoclonal IgG antibody specific for consecutive alignments of β(1-3)-D-glucopyranosyl residues and biotinylated horseshoe crab protein, T-GBP, from Tachypleus tridentatus (24), while the second assay employes a high affinity receptor (galactosyl ceramide) for β(1-3) glucans and a mAb that is described as being specific for complex fungal cell wall β(1-3) glucans (15). The T-GBP-protein—based sandwich ELISA was shown to react readily with β(1-3) glucans including barley β(1-4) (1-3) (24). The other immunoassay, based on the capture agent galactosyl ceramide, was shown to not react with β(1-3) glucan, a glucan which readily reacts with our mabs. Furthermore in that report no analyses were performed with Candida or Cryptococcus whole cells or cell wall fragments.

The presence of β(1-3)-glucans in serum of patients with deep fungal infections may be a useful marker for laboratory diagnosis of these infections. Future investigations will address the usefulness of our mAbs to glucan in an immunoassay-based kit for the rapid detection of β(1-3) glucans in blood samples, in other specimens from patients with invasive fungal infections, or in other body fluids such as mucosal secretions and urine. Moreover, the presence of the β(1-3) or β(1-3) (1-6) glucan epitope on the intact surfaces of both Candida species and non-encapsulated C. neoformans, as seen with A10A, or in cell wall fragments as seen with both A10A and B3B has not been reported earlier regarding activities of monoclonal antibodies directed against β(1-3) glucans.

The antibodies according to the invention can be used for the detection of free, cellwall associated-, and/or cell surface-associated β(1-3) glucans utilizing immunoassays or immunohistology for the laboratory diagnosis of fungal infections. Further, they may be used in immunotherapy.

The antibodies disclosed in the present application may also be used for the detection of airborn mould, or mould present in dust or water, or in any other component. Thus, the antibodies according to the invention may be used for the detection of all kinds of medically important fungi, for example in connection to allergy problems and in the detection of house mould.

### REFERENCES

1. Adachi, Y., N. Ohno, T. Yadomae, Y. Suzuki, M. Ohsawa, and S. Oikawa. 1990. Thermal denaturation of 1-3-beta-D glucans in neutral aqueous solution above 130 degree effect on physicochemical properties. Carbohydr Res 198:111-122.
2. Casanova, M., M. L. Gil, L. Cardenoso, J. P. Martinez, and R. Sentandreu. 1989. Identification of wall-specific antigens synthesized during germ tube formation by Candida albicans. Infect Immun 57:262-71.
3 Fleet, G. H., and D. J. Manners. 1976. Isolation and composition of an alkali-soluble glucan from the cell walls of Saccharomyces cerevisiae. J Gen Microbiol 94:180-92.
4 Hirata, A., Y. Adachi, W. Itoh, M. Komoda, K. Tabata, and I. Sugawara. 1994. Monoclonal antibody to proteoglycan derived from Grifola frondosa (Maitake). Biol Pharm Bull 17:539-42.
5. Hirata, A., M. Komoda, W. Itoh, K. Tabata, S. Itoyama, and I. Sugawara. 1994. An improved sandwich ELISA method for the determination of immunoreactive schizophyllan (SPG). Biol Pharm Bull 17:1437-40.
6. James, P. G., R. Cherniak, R. G. Jones, C. A. Stortz, and E. Reiss. 1990. Cell-wall glucans of Cryptococcus neoformans Cap 67. Carbohydr Res 198:23-38.
7. Jones, J. M. 1990. Laboratory diagnosis of invasive candidiasis. Clin Microbiol Rev 3:32-45.
8. Kapteyn, J. C., L. L. Hoyer, J. E. Hecht, W. H. Muller, A. Andel, A. J. Verkleij, M. Makarow, H. Van Den Ende, and F. M. Klis. 2000. The cell wall architecture of Candida albicans wild-type cells and cell wall-defective mutants. Mol Microbiol 35:601-11.
9. Klis, F. M. 1994. Review: cell wall assembly in yeast. Yeast 10:851-69.
10. Koch, Y., and K. H. Rademacher. 1980. Chemical and enzymatic changes in the cell walls of Candida albicans and Saccharomyces cerevisiae by scanning electron microscopy. Can J Microbiol 26:965-70.
11. Kohler, G., and C. Milstein. 1975. Continuous cultures of fused cells secreting antibody of predefined specificity. Nature 256:495-7.
12. Kondori, N., L. Edebo, and I. Mattsby-Baltzer. 2002. Candida albicans cell wall antigens for serological diagnosis of candidemia. Medical Mycology in press.
13. Kondori, N., L. Edebo, and I. Mattsby-Baltzer. 2002. Circulating B(1-3)-glucan and IgG subclass antibodies to C. albicans cell wall antigens in patients with systemic candidiasis. Submitted.
14. Mattsby-Baltzer, I., Z. Mielniczuk, L. Larsson, K. Lindgren, and S. Goodwin. 1992. Lipid A in Helicobacter pylori. Infect Immun 60:4383-7.
15. Milton, D. K., K. U. Alwis, L. Fisette, and M. Muilenberg. 2001. Enzyme-linked immunosorbent assay specific for (1-->6) branched, (1-->3)-beta-D-glucan detection in environmental samples. Appl Environ Microbiol 67:5420-4.
16. Miyazaki, T., S. Kohno, K. Mitsutake, S. Maesaki, K. Tanaka, and K. Hara. 1995. (1-->3)-beta-D-glucan in culture fluid of fungi activates factor G, a limulus coagulation factor. J Clin Lab Anal 9:334-9.
17. Miyazaki, T., S. Kohno, K. Mitsutake, S. Maesaki, K. Tanaka, N. Ishikawa, and K. Hara. 1995. Plasma (1-->3)-beta-D-glucan and fungal antigenemia in patients with candidemia, aspergillosis, and cryptococcosis. J Clin Microbiol 33:3115-8.
18. Nagi, N., N. Ohno, Y. Adachi, J. Aketagawa, H. Tamura, Y. Shibata, S. Tanaka, and T. Yadomae. 1993. Application of limulus test (G pathway) for the detection of different conformers of (1-->3)-beta-D-glucans. Biol Pharm Bull 16:822-8.
19. Nelson, R. D., N. Shibata, R. P. Podzorski, and M. J. Herron. 1991. Candida mannan: chemistry, suppression of cell-mediated immunity, and possible mechanisms of action. Clin Microbiol Rev 4:1-19.
20. Nguyen, T. H., G. H. Fleet, and P. L. Rogers. 1998. Composition of the cell walls of several yeast species. Appl Microbiol Biotechnol 50:206-12.
21. Obayashi, T., M. Yoshida, T. Mori, H. Goto, A. Yasuoka, H. Iwasaki, H. Teshima, S. Kohno, A. Horiuchi, A. Ito, and et al. 1995. Plasma (1-->3)-beta-D-glucan measurement in diagnosis of invasive deep mycosis and fungal febrile episodes. Lancet 345:17-20.
22. Sanjuan, R., J. Zueco, R. Stock, J. Font de Mora, and R. Sentandreu. 1995. Identification of glucanmanno-protein complexes in the cell wall of Candida albicans using a monoclonal antibody that reacts with a (1,6)-beta-glucan epitope. Microbiology 141 Pt 7):1545-51.
23. Sverremark, E., and C. Fernandez. 1997. Immunogenicity of bacterial carbohydrates: cholera toxin modulates the immune response against dextran B512. Immunology 92:153-9.
24. Tamura, H., S. Tanaka, T. Ikeda, T. Obayashi, and Y. Hashimoto. 1997. Plasma (1-->3)-beta-D-glucan assay and immunohistochemical staining of (1-->3)-beta-D-glucan in the fungal cell walls using a novel horseshoe crab protein (T-GBP) that specifically binds to (1-->3)-beta-D-glucan. J Clin Lab Anal 11:104-9.
25. williams, D. L. 1993. Beta glucans structure and biological function.

1-20. (canceled)

21. A monoclonal antibody reactive with a β(1-3)- and/or a β(1-3) (1-6)-glucan associated epitope in free form, in cell wall fragments or on an intact cell surface.

22. A monoclonal antibody according to claim 21, wherein said β(1-3)- and/or β(1-3) (1-6)-glucan associated epitope is available in cell wall fragments of C. albicans and/or C. neoformans, or on the cell surface of C. albicans, C. parapsilosis, C. krusei, C. glabrata and/or C. neoformans.

23. A monoclonal antibody according to claim 21, wherein said antibody is A10A.

24. A method for the diagnosis of a fungal infection comprising assaying with at least one antibody according to claim 21.

25. A method according to claim 24, wherein said fungal infection is Candida vaginitis or mucocutane candidiasis.

26. A method according to claim 24, wherein said diagnosis is performed on mucosal secretions or urine.

27. Diagnostic kit for the diagnosis of fungal infections comprising a monoclonal antibody according to claim 21.

28. A method for diagnosing fungal infections comprising performing an assay for the detection of β(1-3)-glucans in a sample using a monoclonal antibody according to claim 21, wherein the presence of β(1-3)-glucans indicates a fungal infection in said patient.

29. A monoclonal antibody according to claim 22, wherein said antibody is A10A.

30. A method for the diagnosis of a fungal infection comprising assaying with at least one antibody according to claim 22.

31. A method for the diagnosis of a fungal infection comprising assaying with at least one antibody according to claim 23.

32. A method for the diagnosis of a fungal infection comprising assaying with at least one antibody according to claim 29.

33. A method according to claim 25, wherein said diagnosis is performed on mucosal secretions or urine.

34. Diagnostic kit for the diagnosis of fungal infections comprising a monoclonal antibody according to claim 22.

35. Diagnostic kit for the diagnosis of fungal infections comprising a monoclonal antibody according to claim 23.

36. Diagnostic kit for the diagnosis of fungal infections comprising a monoclonal antibody according to claim 29.

37. A method for diagnosing fungal infections comprising performing an assay for the detection of β(1-3)-glucans in a sample using a monoclonal antibody according to claim 22, wherein the presence of β(1-3)-glucans indicates a fungal infection in said patient.

38. A method for diagnosing fungal infections comprising performing an assay for the detection of β(1-3)-glucans in a sample using a monoclonal antibody according to claim 23, wherein the presence of β(1-3)-glucans indicates a fungal infection in said patient.

39. A method for diagnosing fungal infections comprising performing an assay for the detection of β(1-3)-glucans in a sample using a monoclonal antibody according to claim 29, wherein the presence of β(1-3)-glucans indicates a fungal infection in said patient.
